(19) United States
(12) Patent Application Publication
(10) Pub. No.: US 2002/0131994 A1
(43) Pub. Date: Sep. 19, 2002

(54) Non-irritating formulation for the transdermal delivery of substances

(76) Inventors: Henry B. Schur, Boca Raton, FL (US); Ken Robertson, Boca Raton, FL (US)

Correspondence Address: Robert J. Van Der Wall, First Union Financial Center, Suite 5100, 200 South Biscayne Boulevard, Miami, FL, 33131-2310, US

(21) Appl. No.: 10/042,477

(22) Filed: Jan. 9, 2002

Related U.S. Application Data
(60) Provisional application No. 60/260,786, filed on Jan. 10, 2001.

(51) Int. Cl. A61K 9/70

(52) U.S. Cl. 424/449

(57) ABSTRACT

This invention describes a composition of matter for to facilitate transdermal delivery of a great variety of active substances, especially including high molecular weight drugs such as insulin. Also disclosed is a method of forming the composition of matter and applying topically to patients, especially using a patch. The active substance is initially combined with a biopolymer, which combination is subsequently linked to a base formulation whereby the base formulation aids in the transdermal delivery of the active substance without the skin irritation limitations of other formulations and transdermal methods. The use of a combination of natural herbal, vegetable and animal products combined in this invention with biopolymers permits molecules of higher molecule weight to penetrate the skin in a bioactive form by passive delivery means.

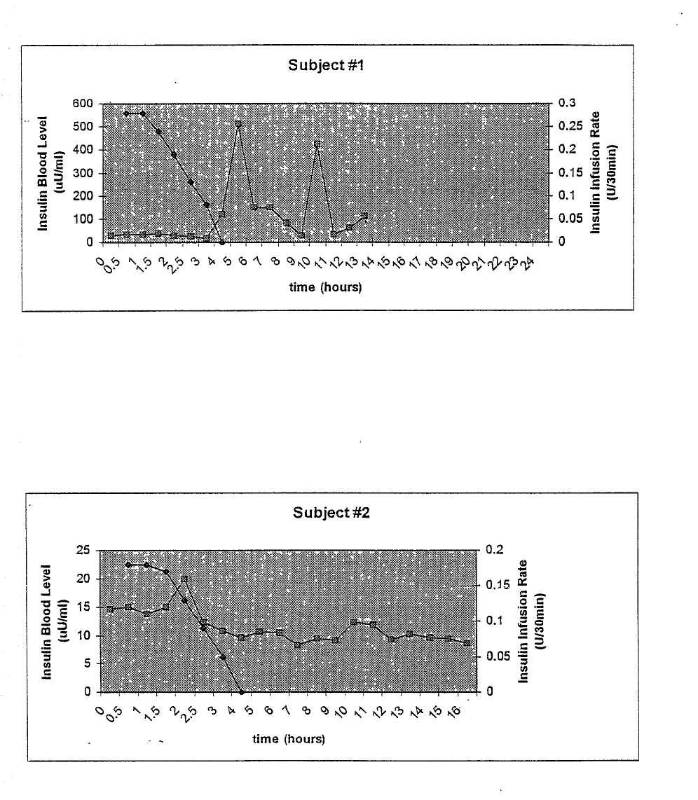

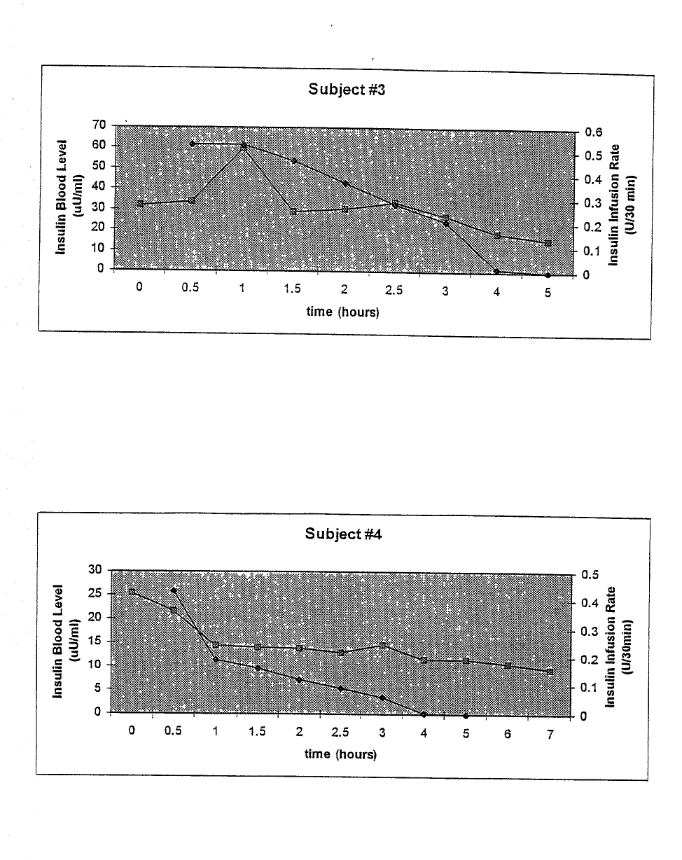

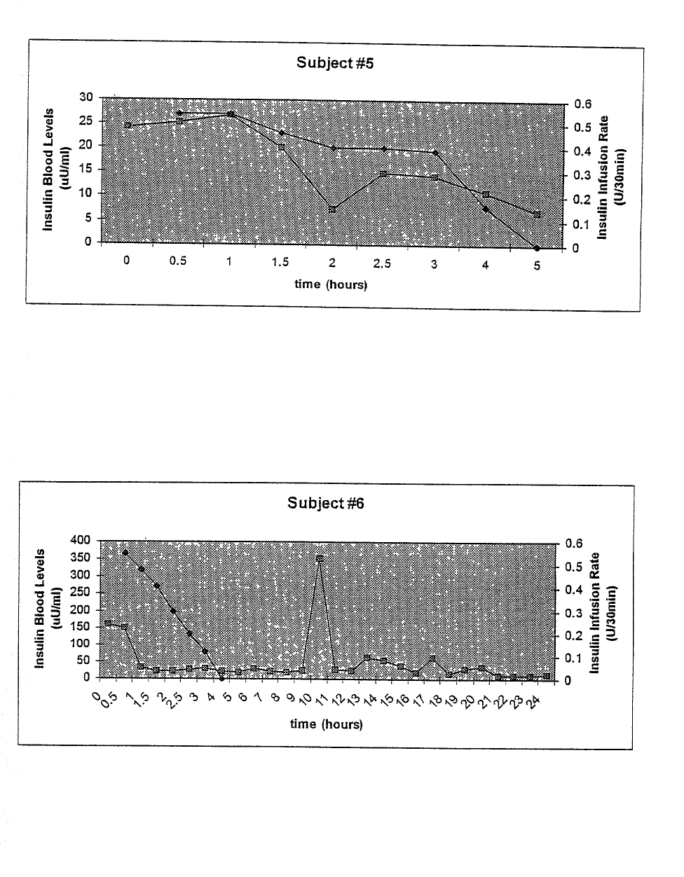

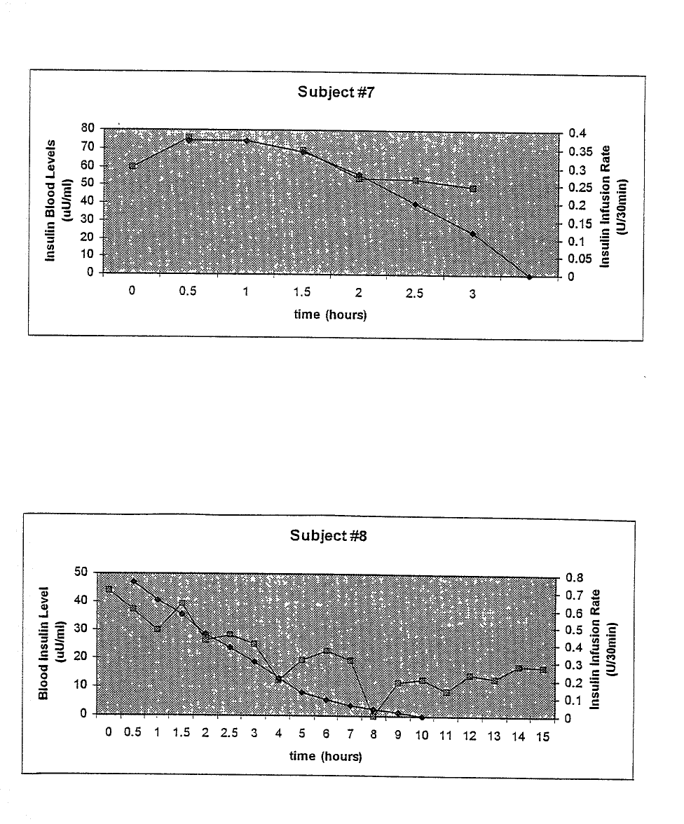

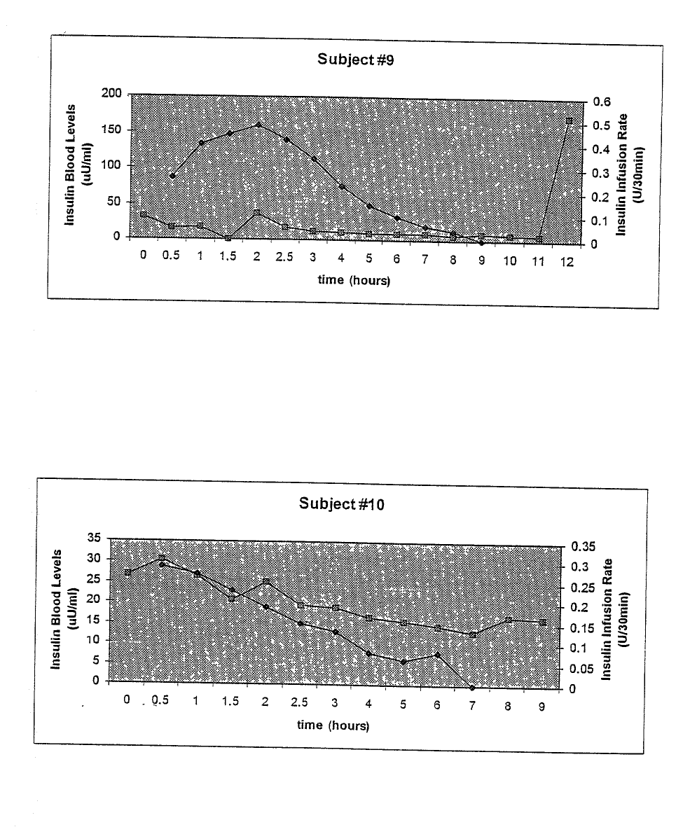

### BACKGROUND OF THE INVENTION

1. Field of the Invention

The present invention relates to the field of a composition of matter for the transdermal delivery of biologically active/chemical substances without the skin irritation limits of other formulations. The use of natural herbal, vegetable and animal products combined in this invention with biopolymers has shown to permit molecules of higher molecule weight to penetrate the skin in a bioactive form by passive delivery means. It also relates to a method of delivery of biologically active/chemical substances that specifically include high molecular weight drugs and also therapeutically-active proteins and numerous other active substances through transdermal delivery. These include, but are not limited to, immunogens, peptides, biophosphonates, cyclosporine and other anti-rejection drugs, anticoagulants, enkelphins, cosmetic ingredients such as collagen and elastin, vitamins, herbal compounds, hormones, chemicals, and the like.

2. Description of Prior Art

Historically, substances were presented to the body through the route of oral ingestion, nasal sprays, intravenously or by injection through or into the skin. Dermal application of substance destined for systemic use have also been used with some success where the molecule being delivered was of small size (<300 Daltons) and of appropriate solubility.

Transdermal drug administration has been touted as a reliable means of achieving continuous dosing of drugs where other means of administration are either discontinuous, labor intensive or where other routes prevent absorption or create inactivation problems. Drugs which are administered through the skin are not subject to first-pass metabolism. A very popular route of administration, oral administration of drugs, however, is subject to first pass metabolism, resulting in incomplete and non-uniform absorption of drugs from the gut. This leads to inconsistent and erratic blood levels of the active substances. In addition, the need for active periodic administration, i.e., three times a day, requires total compliance by the patient in the home or in hospital settings. Due to the aforementioned disadvantages and, last but not least, due to its non-invasive character, transdermal administration has recently become very popular.

Due to the skin dynamics as a living organ and the physical makeup of the skin layers the skin has been shown to behave as a complex barrier to the passage of both simple and complex molecules. The concept of a semi-permeable membrane which follows the physical laws of thermodynamics and concentration/diffusion gradients does not hold up in practice as the molecular size (weight) and configuration increases in both parameters. Additionally, biological factors enter into the complex requirements for a transdermally delivered active substance.

Some chemical/biological molecules are rendered inactive due to the concentration of specific antibodies to them that are resident in the skin. Others can and do cause local irritation which prevents their use based on a medical safety issue. Overcoming both the molecular size and substance irritation problem while maintaining active biological results within the body has been a principal goal of this invention.

Delivery of active substances through the skin layers to a point whereby the substance can be transported to the systemic circulation via the interstitial fluid or microcapillary circulation network can be accomplished using a “patch” device of which there are many designs known to those skilled in the arts or similarly by compounding the active ingredient into an appropriate carrier for direct application to the skin, i.e., cream, lotion, balm, gel, rub and/or ointment. All of these methods of delivering active ingredients to the skin have been used and are in common use today for delivering a variety of substances ranging from drugs to cosmetics (interdermal).

The current invention overcomes the foregoing and other barriers and allows for the transdermal delivery of high (>500 Daltons) molecular weight substances as well as for the use of low (<300 Daltons) molecular weight substances that heretofore were excluded from this mode of delivery due to factors of irritation and/or solubility.

The shortcomings of invasive and metabolized drug administration are obviated by transdermal application of the drugs. A patch is routinely adhered to a clear area of the skin and the drug is continually absorbed through the skin into the bloodstream for systemic absorption. The upper layer of the epidermis (stratum corneum) was previously considered an impenetrable barrier in terms of drug delivery. The advent of skin enhancers has vastly improved the administration of low molecular weight drugs.

The skin is particularly useful as it presents large areas for drug administration, as the skin is the largest organ of the body. The utility of such a mode of administration has been promoted with the discovery and development of a group of compounds that promote transdermal penetration of the various active drugs. Such compounds are known in the art as penetration enhancers or skin enhancers. They are generally characterized to be from the group of monovalent branched or unbranched aliphatic, cycloaliphatic or aromatic alcohols of 4-12 carbon atoms; cycloaliphatic or aromatic aldehydes or ketones of 4-10 carbon atoms, cycloalkanoyl amides of C 10-20 carbons, aliphatic, cycloaliphatic and aromatic esters, N,N-di-lower alkylsulfoxides, unsaturated oils, terpenes and glycol silicates.

These compounds and their specific activity as penetration enhancers, are more fully discussed in the text “Transdermal Delivery of Drugs, A. F. Kydonieus (ED) 1987, CRC Press, and in such patents as Fankhauser, U.S. Pat. No. 4,913,905, Heiber, U.S. Pat. No. 4,917,676, and Sinnreich, U.S. Pat. No. 5,032,403.

As a result of these penetration enhancers, almost any drug, to some degree, can be administrated transdermally. See, for example, such patents as Zaffaroni, U.S. Pat. No. 3,598,122, Zaffaroni, U.S. Pat. No. 3,598,123, Zaffaroni, U.S. Pat. No. 3,742,951, Zaffaroni, U.S. Pat. No. 3,797,494, Zaffaroni, U.S. Pat. No. 3,948,254, Bernstein, U.S. Pat. No. 4,284,444 and Etscorn, U.S. Pat. No. 4,597,961. Examples of such pharmacological active substances include antibacterials such as the penicillins, tetracyclines, second and third generation cephalosporins, chloramphenicol sulfonamides, sedatives and/or hypnotics, such as barbiturates, carbromal, antitussives such as codeine and dextromethorphan, anti-anxiety drugs such as the benzodiazepines including diazepam, buspirone, psychostimulants such as imipramine, amitriptyline and other tricyclic antidepressants, anti-psychotic drugs and tranquilizers such as lithium, chlorpromazine and haloperidol, reserpine, thiopropazate, parkinsonism control agents such as bromotriptine, percolide, the anticholinergics including benzotropine, procyclidine, amantadine (also an antiviral), hormones and hormone antagonists and agonists, including adrenocorticosteroids, insulin, androgenic steroids, estrogenic and pro-gestrogenic steroids, thyroxin and its agonist 5-FU (fluorouracil), tamoxifen, antipyretics and analgesics such as aspirin/acetaminophen and other non-steroidal anti-inflammatory drugs (NSAID), analgesics based on morphine, morphine antagonists, vasodilating agents such as nitroglycerine, isorbide dinitrate, alpha and beta-blockers and other cardioactive drugs, antimalarials, antihistamines and anticholinergics including atropine, hyoscyamine or methscopalomine (for motion sickness), weaning agents such as nicotine (for tobacco addiction), and antiasthmatic bronchodilators such as formoterol, and combinations of such pharmaceutical active substances.

Of course, while feasible, not all of these active substances have yet been completely tested for efficacy by transdermal administration but many are under vigorous scrutiny. Other active substances at this time are not economically viable for such administration, as the cost of full safety testing is too great for the specific number of patients involved.

It is noted, in particular, that high molecular weight drugs, including proteins and peptides have not had many successes in terms of passive delivery of drugs with a minimum degree of irritation. Many of the attempts to deliver high molecular weight substances have been achieved mostly through aggressive means.

Johnson, U.S. Pat. No. 5,947,921, teaches and elucidates possible mechanisms of skin permeant enhancement using both chemical and physical means. Johnson also discusses the need for preventing skin irritation during transdermal drug delivery due to either/or penetrant chemicals or drug actives. It describes the use of sonophoresis as a means to provide delivery of proteins and peptides through the skin with the use of chemical enhancers. In addition, the sonophoresis may also rely on other aggressive assists such as mechanical or osmotic pressure, magnetic fields, electroporation, or iontophoresis.

D'Angelo, et al., U.S. Pat. No. 5,614,212, describes delivery of drugs ranging from 500 to 6000 Daltons and encapsulation of a drug in polyvinylpyrrolidone (PVP) in a microsphere composed of alginate and optionally a cross-linked alginate. D'Angelo '212 does not teach the use of PVP as a “conditioner” nor does it teach a pre-use incubation phase whereby PVP acts as a “binding” agent to allow the active drug to be incorporated into a suitable formula for the non-irritating delivery of active components. The present invention improves and expands in new art the use of PVP as an excipient and in unanticipated ways over D'Angelo '212, in combination with other excipients to achieve a non-encapsulated, non-irritating drug delivery system. D'Angelo '212 also excludes PVP as the preferred enhancer for the delivery of insulin and thus specifically calls for the use of Azone™. The present invention teaches that PVP is compatible with insulin when used in the stated new method elucidated herein.

D'Angelo, et al., U.S. Pat. No. 6,024,975 describes a way to deliver high molecular weight drugs by transdermal administration, consisting essentially of a drug having a molecular weight ranging from 50 Daltons to 25,000 Daltons, a polymer which is polyvinylpyrrolidone and an optional gelling agent. The patent claims the delivery of Calcitonin and Insulin and one that can be achieved by optionally adding electronic means to enhance absorption, microspheres and solubility enhancers chosen from a group including acetamide, N,N-dimethylacetamide, N,N-diethylacetamide, C.sub.10-C.sub.20 alkanoylamides, 1-N-C.sub.10-C.sub.20-alkylazacycloheptan-2-one, N-2-hydroxyethylacetamide, dimethyl sulfoxide, salicylates, polyalkylene glycol silicates, and mixtures thereof. In similar manner to D“Angelo '212, D'Angelo '975 does not teach the use of PVP as a “conditioner” nor does it teach a pre-use incubation phase whereby PVP acts as a “binding” agent to allow the active drug to be incorporated into a suitable formula for the non-irritating delivery of active components. The present invention improves and expands in new art the use of PVP as an excipient and in unanticipated ways over D'Angelo '975, in combination with other excipients to achieve a non-encapsulated, non-irritating drug delivery system.

Gertner, U.S. Pat. No. 5,707,641, teaches a pre-treatment step for insulin which consists of allowing the insulin hexamer to dissociate into a dimer or monomer over a 30 day period at temperatures over 4° C. and preferably over 20° C. This step effectively reduces the molecular weight from approximately 6000 to 3000-4000 Daltons and thus make its delivery art similar to many others in the field who have shown transdermal delivery of molecules below 4000 Daltons. The present invention does not rely on the reduction of the molecular weight of the drug active to achieve systemic delivery through the skin. The present invention teaches a new method of incubation of the active drug with a compound that will act as a “combining” agent and allow for the drug to attach to the excipient/penetrant formulation regardless of molecular weight. Gertner does not teach or suggest the addition of any compound to the active drug during his decomposition stage. The present invention utilizes an incubation period to allow the gentle combining of the “coupling” agent with the active drug which is carried out in a short time period (7 days) and can be accomplished at 4° C. for product stability. Further, those skilled in the arts will appreciate the present invention's ability to be adapted to a wide variety of compounds that by their nature will not de-polymerize upon standing as a way to lower their molecular weight. In fact many drugs if put through the Gertner process would lose their efficacy.

Foldvari, U.S. Pat. No. 5,718,914, broadly describes the delivery of topical agents through the use of liposomes. The liposomes are described to be particulates able to pass through membranes having pores of 0.1 to 500 microns. The formulations are intended to be delivered through a patch in a reservoir behind the above described membrane. It also teaches the construction of a suitable patch to contain a composition of matter instant to the present invention along with patent literature references for same all of which are incorporated herein by reference.

Skinner, U.S. Pat. No. 5,449,670, teaches that there may be a “conditioner” effect in using some pyrrolidone compounds to aid in the delivery of active components below the 4000 molecular weight and teaches that preferably the molecular weight should be below 3500 molecular weight. The present invention teaches that specifically a vinyl pyrrolidone when incubated at specified conditions with an active compound can, when further formulated into the present invention, deliver drug compounds in excess of 5000 molecular weight. This being a great improvement over Skinner in that most new therapeutic drugs being developed are of large molecular weights (i.e., synthetic insulin, growth hormone, etc.). The present invention advances Skinner and teaches a new method and formula for using PVP in a heretofore unanticipated way even by those skilled in the arts.

Clement, U.S. Pat. No. 5,208,028, teaches the use of a multi-step emulsion mixture process. It uses particulates created from a combination of aqueous dispersion of fatty acids, fatty alcohols, oils, basic compounds such as triethoxylamine, saccharides, alginates, chitin, metal salts, structural polymers such as carboxypropyl cellulose or xanthan gums. All the combined components are emulsified into an aqueous dispersion which is then used for topical administration of cosmetic ingredients. However the present invention is not dependent on the cross linking of the Clement emulsion to achieve its result. Further Clement requires that “capsules” be formed to protect or isolate the active component prior to use. The present invention is an improvement over Clement as no “activation” is required and a mechanical dispenser (pump) is not required for the product to achieve its stated goal.

Ghosh, U.S. Pat. No. 5,431,924, teaches the fractionation of emu oil into a biologically active substance having claimed therapeutic value. Furthermore, it is claimed that to obtain this value the product must be placed in a suitable carrier for transdermal delivery. The present invention teaches that emu oil can be used in its unfractionated state as an excipient and protectorant of active pharmaceutical ingredients. The use of emu oil as an example of a refined avian oil for an excipient in the compounding of a transdermal delivery system is a new and significant advantage over previous saturated fatty acid emulsification excipients. The present invention, on the other hand teaches and claims that the use of emu oil in a new and novel emulsification/transport material when combined with other natural oils effectively aids in the transport of active drugs across the dermis while reducing inflammation at the application site. Ghosh does not teach the use of emu oil as a transport vehicle or as an emollient or as a humectant all properties that are utilized in the present invention as an aid in the non-irritation delivery of active ingredients.

Fein, et al., U.S. Pat. No. 5,472,713, describes a method of lowering cholesterol or triglycerides through the oral, parenteral, enteral, rectal and systemic administration of 2-10 mls of emu oil per day.

### SUMMARY OF THE INVENTION

Bearing the mind the foregoing, a principal object of the present invention is to provide a composition of matter comprising an active substance combined with a biopolymer, which combination is linked to a base formulation whereby the base formulation aids in the transdermal delivery of the active substance.

A further object of the invention is to utilize a composition of matter wherein this composition may be applied to the skin as a topical treatment such as a cream, lotion, balm, gel, rub and/or ointment.

Another object of the invention is the use of a combination of biopolymers and natural herbal and animal products in a composition of matter to transdermally deliver substances without irritation.

A related object of invention is to specifically include in the group of active substances to be administered not only drugs, but such as immunogens, peptides, biophosphonates, cyclosporine and other anti-rejection drugs, anticoagulants, enkelphins, cosmetic ingredients such as collagen and elastin, vitamins, herbal compounds, hormones, chemicals, and the like.

Another principal object of the invention is to provide a viable system and method for the transdermal administration of active substances including high molecular weight drugs, such as insulin, of upward of 150 Daltons with a polymer skin enhancer and an ingredient that minimizes inflammation.

It is a related object of the present invention to provide a method of transdermally administering an active substance that may be a high molecular weight drug, which in summary includes applying to skin of a patient a polymer skin enhancer, and applying to the skin of the patient a drug active (15% or more of the system) having a molecular weight of above 150 Daltons and preferably above 500 Daltons. The preferred skin enhancer is polyvinylpyrrolidone.

Another object of the invention is pre-incubation of an active substance, such as (but not limited to a high molecular weight drug) with a biopolymer under conditions which are suitable for the mutual solubilization of the active substance and the polymer while maintaining the desired biological activity of the active substance.

Another object of the invention is for the composition to be applied using a transdermal patch.

A similar object of the invention is to provide a process whereby a biopolymer is combined with an active substance to be delivered and then linked to a base formulation which aids in the delivery of said active substance.

Another object of the invention is the combination of biopolymer and base formulation which prevents skin irritation caused by the active substance in the present composition of matter and method.

A further object of the invention is a system of adjusting the hydrophobic/lipophobic nature of the inventive composition of matter to allow various solubility of active substance with which it is intended to be used in the method of the present invention.

An additional related object of the inventive method is to provide that biopolymer is incubated with active substance to initiate binding reaction prior to combination with other ingredients.

One more object of the invention is to provide that a base formulation consisting of both aqueous and non-aqueous components is combined into a homogeneous mixture with adjustable viscosity.

A further object of the invention is the use of an organic solvent of high volatility in the preparation of the base formula that evaporates off during the homogenizing process.

Another object of the invention is to select the biopolymer from the group of polymers represented by polyvinylpyrroliodone, alginates, chitin, collagen, Elastin and similar materials.

An additional object of the invention is to include aloe vera in the group from which the herbal extract is selected.

A further object of the invention is to select the vegetable component from the group including the natural extract of: marcrocystis sp., oil of coconut, corn oil, soy oil, almond oil, and the like.

One more object of the invention is to select the natural animal products from the group that includes the oil from the avian species: Emu, chicken, turkey, ostrich, glycerol, etc.

A further object of the invention is to include in the acceptable solvents ethyl alcohol, isopropyl alcohol, acetone, and methanol.

Other objects and advantages will be apparent to those skilled in the art.

With the foregoing objects of the invention in mind there are provided, in accordance with the invention, compositions of matter that include a polymer system for effecting delivery of high molecular weight an active substance by transdermal administration. The system includes at least 15% by weight of an active substance having a molecular weight of more than 150 Daltons, a polymer which is preferably polyvinylpyrrolidone, a weight of the polymer being 7 to 35% by weight of the active substance, and an optional gelling agent having from 0 to 20% by volume of the system. The gelling agent can be chosen from the group consisting of alginates, chitin, collagen, elastin and the like.

The active substance of the polymer system may be a hormone, i.e., calcitonin or insulin or proteins such as heparin. (Animal studies using heparin have been performed with great success.) The polymer may be a biocompatible polymer of the pyrrolidone group, e.g. polyvinylpyrrolidone (PVP). If PVP is used it may have a K-value of K-10 or K-40. Other polymers with solubility characteristics similar to polyvinylpyrrolidone may also be considered.

The ingredient that is capable of inhibiting inflammation is on that is obtained from the natural animal products consisting of the oil from the avian species such as emu, chicken, turkey, or ostrich and can represent anywhere from 1 to 20% of the composition, most preferably with the ingredient approaching 8-10% of the total composition.

The finished composition can be applied as a topical treatment wherein the compound is unit dose dispensed from a suitable package and spread on the skin or alternatively can be incorporated into a transdermal delivery patch of a standard design and known to those familiar with the art and then applied to a selected area of the body. The composition may be fashioned as a transdermal patch, cream, lotion, balm, gel, rub and/or ointment. Topical use may be applicable in animals where a physical patch would be difficult to maintain in contact with the skin.

In accordance with another major aspect of the present invention there is provided a method that includes pre-incubation of an active substance, such as (but not limited to, a high molecular weight drug, e.g., insulin) with a biopolymer under conditions which are suitable for the mutual solubilization of the active substance and the polymer while maintaining the desired biological activity of the said substance. This pre-incubation period is a required step to assure that the interaction of the active substance and the polymer have occurred. This step can be done at temperatures between 4 degrees Celsius and 37 degrees Celsius for times ranging from a few minutes to as much as 30 days.

The pre-imbibed polymer is then added to a solvent rich formulation and homogenized in a suitable high shear homogenizer during which the solvent is removed through appropriate means. The solvent used for this can is one that preferably is volatile and can be chosen from the following group such as ethyl alcohol, isopropyl alcohol, acetone, or methanol. The solvent rich formulation can contain solvents that are polar and non-polar, aqueous and non-aqueous or any combination thereof. The selection of solvent systems will vary as the chemical/physical properties of the substance to be delivered are varied. In the example cited an ethanol/water solvent system is used.

The viscosity of the finished formulation is adjusted during mixing to produce the desired consistency for the intended use. Since the formulation exhibits classic thixotropic properties rest time must be incorporated into the process to avoid viscosity variations.

The non-aqueous components of the formulation consists of an emulsification of the carrier ingredients by first incorporating the non-aqueous soluble components with the biopolymer under high shear mixing and then adding the aqueous materials again under high shear mixing. Some of the non-aqueous components can be chosen from the group consisting of macrocystis sp., oil of coconut, corn oil, soy oil, or almond oil.

The percentage of active compound required will vary depending on the pharmocodynamics, delivery rate, solubility, dose requirements, bioavailability, and other factors. Active materials can represent from as low as 0.01% to as high as 60% of the total composition. Compounds with molecular weights from the low, i.e., 150-180 Daltons, to as high as many thousand (6000) Daltons can be used with this delivery system. The upper limit on molecular weight has not been established but can theoretically be as high as 25,000 Daltons.

### DETAILED DESCRIPTION OF THE PREFERRED EMBODIMENT

As required, detailed embodiments of the present invention are disclosed herein; however, it is to be understood that the disclosed embodiments are merely exemplary of the invention which may be embodied in various forms. Therefore, specific functional details disclosed herein are not to be interpreted as limiting, but merely as a basis for the claims hereto appended and as a representative basis for teaching one skilled in the art to variously employ the present invention in virtually any appropriate circumstance.

The preferred embodiment of this invention has been derived from both animal experimentation and human clinical experimentation. A number of specific examples are presented hereinafter based on the results using insulin (6000 Dalton molecular weight) patches with Type I insulin dependent diabetics.

The patch was constructed using standard materials provided by the manufacturer (Minnesota Mining & Manufacturing-3M Transdermal Division). The patch was a reservoir style with the dimensions of 25 mm diameter×5 mm for the reservoir and an overall dimension of 40 mm×40 mm. The construction was standard and known to those skilled in the arts of transdermal patch design. The patches were filled with 2.8-2.9 grams of test compound per patch. The integration of the active ingredient into the total compound starts by first determining the dose requirements per 24 hours and the solubility of the active ingredient in an acceptable solvent. The Solvent of choice is water for insulin. Other factors such as pH. lipid affinity and temperature stability also must be considered in adjusting the first stage of the process and composition.

Drug active skin irritability must also be considered when selecting the biopolymer for first step incubation. Insulin, which has a 1) mild to moderate skin irritation factor, 2) high solubility in water, 3) pH requirements of 5.5 determined the selection of polyvinylpyrrolidone (PVP) as the biopolymer best able to act as linking/coupling agent and protecting agent for this process.

The insulin (Novo, Denmark) was placed in a sterile glass container (20 ml of a 500IU commercial preparation) and then stirred slowly using a magnetic stir plate. To this was added slowly 25 mg of PVP K-30 (Sigma Chemical, U.S.) while maintaining the stirring. The container was then covered tightly. The mixture was left to incubate at room temperature (20 C.) for 7 days. After the incubation time elapsed the resultant product was stored at 4 C. until used in Part II of the process.

Part II of the process includes pre-manufacture of several of the components.

The PVP base used in Part II is made by dissolving PVP K-40 (Sigma Chemicals, U.S.) in an ethyl alcohol solution. A 60% concentration of the PVP is dissolved in 95% ethyl alcohol (VWR Scientific, U.S.) using high shear mixing to start and then continuing to mix at low speed for 48 hours. Precautions must be taken to avoid solvent evaporation while making this ingredient. After mixing and solution is complete the mixture must “rest” for an additional 72 hours to allow the compound to stabilize. Each a multi-solvent “flash off” system using carious phase replacement technology which is unique to this invention.

The Kelgin HV (Monsanto, U.S.) is prepared by slowly adding the powdered Kelgin HV to distilled water to make a 5% solution using a high shear mixer. The temperature of the product must not exceed 45 C. during the mixing process. The addition of methylparaben (1:10,000) to the Kelgin mixture will assure its biological safety and will act as a safe and effective preservative for the final product. The Kelgin mixture should stand at room temperature for 72 hours to allow for complete hydration of the polymer and stabilization.

Preparation of the final composition must proceed in such a manner as to preserve the purity and efficacy of the compound by using aseptic techniques throughout.

The final composition of compound is made as follows:

To the required calculated percentage of PVP base previously made (17%) add the K-oil stock solution (Texas EMU Cooperative, U.S.) (8.4%) which has been stored at 37 C. prior to use and emulsify with a high shear mill. After emulsification is complete add the coconut oil (Spectrum Chemicals, U.S.) (2.4%) and the glycerin (Spectrum chemicals, U.S.) (8.7%) and emulsify as previous. Check for stability of emulsification at this point. After a 30 minute stability has been achieved add the aloe (Natural High Products) (5%) slowly with rapid stirring. When the aloe is fully incorporated into the mixture add the previously made Kelgin HV (17.2%) and re-emulsify in colloid mill. Allow this compound to rest for 30 minutes and then add the previously prepared insulin composition (34%) and pass through homogenizer/emulsifier mixer again. Allow final product to return to its normal rheological state for 30 minutes prior to filling patch reservoirs.

Final product can be bulk stored in sealed containers at 4 C. for several weeks but must be remixed prior to filling. Patches are filled with 2.8 grams of material and exposed well covered with release liner (3M, U.S.) placed in multi-laminate barrier foil pouches and sealed until use.

This composition has been shown to be stable at room temperature for at least 10 days and several months when stored at 40 C., when packaged as described.

The filled patches are used by removing them from the foil patch immediately before use, removing the release liner to expose the filled patch well and adhesive. The patch was placed on the inner forearm at the wrist for observation during the clinical trials.

The following examples demonstrate the best mode that has been obtained to date for passive delivery of high molecular weight substances in what appears to be non-inflammatory composition for the compounds being tested.

### EXAMPLE 1

The following example of optimized formulation for insulin was compounded at room temperature by using the steps shown:

1. Insulin/polymer combination was prepared by adding PVP (K40 Sigma Chemicals, US) to Humulin R (500 U/ml) and incubating for 7 days with stirring.

2. PVP Solution was prepared by dissolving under high shear conditions PVP (K-40) in ethyl alcohol in a ratio of 60:40 and allowing this solution to rest at least 5 days prior to use.

3. Prepare the Kelgin Solution using Kelgin HV (Monsanto) in distilled water in a ratio of 5:95 using a high shear mixer. Add to this a preservative such as methylparaben up to a 1% concentration to prevent bacterial and mold growth.

4. Proceed to combine the PVP Solution with Emu Oil (Texas Emu Cooperative), coconut oil (Spectrum Chemicals) and glycerin (Spectrum Chemicals) with a homogenizing mill.

5. To the product of step 4 add Kelgin Solution and Aloe (Natural High Products) mix with homogenizing mill.

6. Add the insulin/polymer to product of 5 and remix.

7. Let product 6 stand for 30 minutes and then disperse into patches for use.

8. Store patches in a sealed barrier foil pouch (Kenpak) until use.

The composition of all the components in the above formulation in their combined form resulted in the composition as is shown on Table 1. This composition contained a theoretical amount of 34% by weight of insulin. The resulting cream was fashioned into patches which were tested in rabbit and human studies. The rabbit and human studies both demonstrated the ability of the patches to absorb insulin into the bloodstream shortly after application of the patches.

TABLE 1
Composition of Transdermal Insulin Formulation

| Ingredient | grams | % of Final Formulation |
|---|---|---|
| PVP Solution | 5.1 | 17 |
| PVP K-40 | 0.1326 | 1.3 |
| Emu-Oil | 2.61 | 8.4 |
| Kelgin Solution | 5.25 | 17.2 |
| Coconut Oil | 2.61 | 8.4 |
| Glycerin | 2.61 | 8.7 |
| Aloe | 1.5 | 5 |
| Insulin | 10.2 | 34 |

Prior to testing in humans, the above formulation without the drug was evaluated to determine if it would produce allergic skin reactions following epicutaneous application to albino guinea pigs, otherwise known as the Buehler Sensitization Test. The study was undertaken by Toxicon Corporation, Bedford, Mass. under study # 00-2745-G2. The conclusions of the study indicated that the above formulation is not considered to be a skin sensitizer since none of the test animals exhibited erythema and/or edema at the challenge exposure (36 hours) following an induction phase (6 hours/day; 5 days/week, 3 consecutive weeks).

In another study, Toxicon performed an Acute toxicity in Rabbits—45 hours, under study #00-2745-G1. Assessments including clinical observations and body weight measurement, hematological and clinical chemistry status, necropsy and organ weight determinations, and histopathological analysis of selected tissues. The results indicated that the transdermal product did not elicit any acute toxicity at a dose of 5 grams/animal, as evidenced by the lack of any significant differences in any of the assessed parameters compared to the control animals.

A Phase I clinical study was conducted on and insulin patch under the Direction of Jay Skyler, M.D. of the University of Miami Jackson Memorial Medical Center under IND #59,542.

The experimental protocol used to verify the preferred embodiment of this invention was essential to the development of a formulation and process which will function as desired. All human patients (diabetic) were confirmed HgAlc negative indicating that they did not produce insulin form islet cells and thus any measurable insulin in their blood was from therapeutic injection or the patch. A technique called “insulin clamping” was used whereby a test patient was placed on a I.V. drip of insulin in one arm and an I.V. drip of glucose in the other arm. Glucose levels were monitored on an average of 15 and 30 minutes using an indwelling catheter and withdraw blood samples. Insulin levels were determined by routine clinical laboratory method in an approved third party independent clinical laboratory. The Standard clearance time for insulin I.V. is considered to be <20 minutes. All insulin measured in the circulating blood after the insulin infusion was terminated for >20 minutes was due to the patch delivery. The graphs described below show the results of Type I human test subjects and plot the insulin infusion vs. insulin level vs. time. All insulin values after the insulin infusion rate+20 minutes represent the insulin that was delivered from the patch.

The study showed conclusively that insulin was delivered from the patch into Type I diabetics that are unable to produce their own insulin (these are shown in the graphs labeled Subject 1-10, representing each of the patients tested). In addition, Dr. Skyler suggested the following from his observations of the trial:

the patch was well tolerated, and no adverse events were attributable to the patch

there was clear evidence of insulin absorption/increased plasma insulin levels attributable to the patch

there was sufficient promise from this study to further develop a patch that can offer sustained and reproducible insulin delivery

The following graphs of subjects 1-10 show the results of Infusion of Insulin and Blood Insulin levels of ten type I diabetic patients who were all given patches. The infusion was intended to maintain the patients at the beginning of the patch trial. Once the infusion was removed, it was evident in all 10 patients that the measured blood level of insulin remained measurable up to the last data time point taken.

As a reference in Subject #1, note the constant decrease of Insulin Infusion up to time point 4 hours, afterwards and up to time point 8.5 hours, there remains measurable amounts of Insulin remaining in the bloodstream that can only be coming from the patch the patient was wearing. A similar trend is seen for the next 9 subjects. It is very apparent, insulin is maintained from the patch once the Insulin IV is removed in subjects 2, 4, 6, 8, 9, and 10.

In addition to the above human studies, 3 versions of Rabbit studies were conducted. In all these studies, not only were there measurable levels of insulin being delivered into the bloodstream of the rabbits, there was also noted no obvious sign of irritation at the site of administration of the patch at up to 4 hours of administration.

### EXAMPLE 2

Following the procedure detailed in Example 1, the below formulation was prepared with the active substance being bisphosponate.

TABLE 2
Composition of Transdermal Bisphosphonate Formulation

| Ingredient | grams |
|---|---|
| PVP Solution | 5.1 |
| PVP K-40 | 0.1326 |
| Emu-Oil | 2.61 |
| Kelgin Solution | 5.25 |
| Coconut Oil | 2.61 |
| Glycerin | 2.61 |
| Aloe | 1.5 |
| Bisphosphonate | 3000 Units |

### EXAMPLE 3

Following the procedure detailed in Example 1, the below formulation was prepared with the active substance being cyclosporin.

TABLE 3
Composition of Transdermal Cyclosporin Formulation

| Ingredient | grams |
|---|---|
| PVP Solution | 5.1 |
| PVP K-40 | 0.1326 |
| Emu-Oil | 2.61 |
| Kelgin Solution | 5.25 |
| Coconut Oil | 2.61 |
| Glycerin | 2.61 |
| Aloe | 1.5 |
| Cyclosporin | 0.25 |

### EXAMPLE 4

Following the procedure detailed in Example 1, the below formulation was prepared with the active substance being heparin.

TABLE 4
Composition of Transdermal Heparin Formulation

| Ingredient | grams |
|---|---|
| PVP Solution | 5.1 |
| PVP K-40 | 0.1326 |
| Emu-Oil | 2.61 |
| Kelgin Solution | 5.25 |
| Coconut Oil | 2.61 |
| Glycerin | 2.61 |
| Aloe | 1.5 |
| Heparin (Low MW) | 1.0 |

The above examples 2, 3, and 4 were all tested in patch configurations as a 1″ diameter reservoir system. Each formulation was individually tested on Juvenile Pigs weighing 3-5 kgs. The animals were intubated with an external jugular venous line. Prior to placement of the transdermal patch an initial baseline sample of blood is taken. Thereafter, 3 cc of blood were taken at 30 min intervals after initial placement of the patch. Sampling was done every hour for up to 6 hours.

In all cases there was evidence of absorption of the drugs into the bloodstream via the patch with no apparent irritation being noted at the site of application.

While the invention has been described, and disclosed in various terms or certain embodiments or modifications which it has assumed in practice, the scope of the invention is not intended to be, nor should it be deemed to be, limited thereby and such other modifications or embodiments as may be suggested by the teachings herein are particularly reserved especially as they fall within the breadth and scope of the claims appended hereto.

What is claimed is:

1. A composition of matter for transdermal delivery of an active substance comprising: the active substance in combination with a biopolymer, which combination is linked to a base formulation whereby the base formulation aids in the transdermal delivery of the active substance.

2. The composition of claim 1 in which the biopolymer is selected from the group of polymers represented by polyvinylpyrroliodone, alginates, chitin, collagen, and elastin.

3. The composition of claim 1 in which the biopolymer is preferably polyvinylpyrroliodone.

4. The composition of claim 1 in which the base formulation includes an ingredient that minimizes skin inflammation.

5. The composition of claim 1 wherein the base formulation further includes at least one of natural herbal, vegetable and animal products.

6. The composition of claim 5 in which the vegetable product is selected from the group including the natural extract of: marcrocystis sp., oil of coconut, corn oil, soy oil, and almond oil.

7. The composition of claim 5 in which the animal product is selected from the group that includes glycerol and oil from avian species such as emu, chicken, turkey, and ostrich.

8. The composition of claim 5 in which the herbal product is selected from the group that includes aloe vera.

9. The composition of claim 1 wherein the base formulation includes solvents selected from the group that are aqueous, non-aqueous, polar, and non-polar which are combined to form a homogeneous mixture with adjustable viscosity.

10. The composition of claim 1 wherein the base formulation further includes at least one solvent selected from the group of ethyl alcohol, isopropyl alcohol, acetone, and methanol.

11. The composition of claim 1 which facilitates transdermal delivery of insulin without skin irritation.

12. A method for enhancing transdermal delivery of an active substance comprising:
combining the active substance with a biopolymer;
creating a base formulation that includes a least one ingredient that minimizes skin inflammation;
linking the biopolymer and active substance combination with the base formulation to form a composition of matter; and
applying the composition of matter topically to a patient.

13. The method of claim 12 in which the step of combining the active substance with a biopolymer includes a pre-use incubation phase wherein the biopolymer acts as a binding agent to enhance transdermal delivery of the active substance after the active substance and biopoolymer are linked with the base formulation.

14. The method of claim 12 in which the step of linking the biopolymer and active substance combination with the base formulation to form a composition of matter further comprises:
adding the biopolymer and active substance combination to a solvent rich base formulation to create a mixture; and
homogenizing the mixture during which the solvent is removed.

15. The method of claim 12 in which the step of applying the composition of matter topically to a patient includes at least one of use of a transdermal patch, cream, lotion, balm, gel, rub and ointment.

16. The method of claim 12 wherein the composition of matter includes a biopolymer, and at least one of natural herbal, vegetable and animal products.

17. The method of claim 12 wherein the composition of matter is adjusted for a hydrophobic/lipophobic nature of the composition to allow integration of substances with various solubility characteristics.

18. The method of claim 12 wherein the base formulation includes solvents selected from the group that are aqueous, non-aqueous, polar, and non-polar which are combined to form a homogeneous mixture with adjustable viscosity.

19. The method of claim 12 wherein an organic solvent of high volatility selected from the group of ethyl alcohol, isopropyl alcohol, acetone, and methanol is used in preparing the base formulation.

20. The method of claim 12 which facilitates transdermal delivery of insulin without skin irritation.
